(19) United States
(12) Patent Application Publication
(10) Pub. No.: US 2022/0204906 A1
(43) Pub. Date: Jun. 30, 2022

(54) RESEALABLE MICROBIAL CULTURE AND OBSERVATION DEVICE

(71) Applicant: BIOMED DIAGNOSTICS, INC., White City, OR (US)

(72) Inventors: John F. Antiabong, Medford, OR (US); Josiah L. Fast, Central Point, OR (US)

(73) Assignee: BIOMED DIAGNOSTICS, INC., White City, OR (US)

(21) Appl. No.: 17/516,898

(22) Filed: Nov. 2, 2021

Related U.S. Application Data
(60) Provisional application No. 63/131,693, filed on Dec. 29, 2020.

(51) Int. Cl. C12M 3/00; C12M 1/00; C12M 1/12

(52) U.S. Cl. CPC C12M 23/54; C12M 23/22; C12M 23/38; C12M 23/34; C12M 37/02; C12M 29/04; C12M 29/20; C12M 37/04; C12M 23/44

(57) ABSTRACT

A resealable microbial culture and observation device provides a growth platform for the growth, transportation, and inspection of a biologic organism. The device provides a tray having at least one growth medium depression for containing a growth medium and a biologic, at least one anerobic member depression for containing an anerobic member that creates an anerobic environment for the biologic, and a vent to enable filtered gas exchange with the ambient environment, so as to supplement the microbial growth conditions in the growth medium depression during single use or in stacked position of multiple devices; and further having a resealable label with a nontransparent portion for labeling, and a transparent portion for viewing the biologic.

### CROSS-REFERENCE TO RELATED APPLICATIONS

This application claims priority from U.S. Provisional Application Ser. No. 63/131,693, entitled “Resealable Microbial Culture and Observation Device”, filed on Dec. 29, 2020, which application is hereby incorporated herein by reference in its entirety.

### FIELD OF THE INVENTION

The present invention relates generally to a microbial culture observation device. More so, the present invention relates to a resealable microbial culture and observation device that allows growth, transportation, and inspection of microbial growth characteristics.

### BACKGROUND OF THE INVENTION

A microbial or cell-culture device alternatively known as a Petri dish or Petri plate, which is usually a round, shallow, flat-bottomed, glass or plastic dish with a vertical side that cooperates with a similar, slightly larger structure which forms a loosely fitting lid. The purpose of the Petri dish is to provide a controlled environment for selectively growing microbes. The dish is sterilized and designed to maintain a sterile environment inside while freely exchanging gases, normally air, with the outside environment. Biologists generally use petri dishes to hold growth medium in which cells or microorganisms can be cultured.

Although the structure of a Petri dish is quite simple, it must perform its intended function as efficiently and inexpensively as possible. Ideally, the Petri dish should be a shallow rigid container which is easily managed by a technician with respect to manual placement and removal of the cover member. Further, the dish must be susceptible to stacking, the stacked dishes interrelating with one another sufficiently to permit convenient transport in the stacked configuration.

Numerous attempts have been made and several prior art devices are known for variety of Petri dishes of microbial culture devices. Even though these innovations may be suitable for the specific purposes to which they address, however, they would not be as suitable for the purposes of the present invention.

For example, U.S. Pat. Nos. 6,429,008 and 5,955,344 to Copeland et al. disclose a microbial culture dish which can be inverted to produce an anaerobic environment. A seal that traps the gas in the headspace between the media surface and the inside of the dish top. An oxygen reducing agent is incorporated into the media present in the dish to create an environment suitable for growing and maintaining anaerobic microorganisms.

U.S. Pat. No. 5,858,770 to Perlman relates to cell culture plates that contain cells incubated in a liquid medium held in sample wells, wherein the plate is sealed with a waterproof adhesive sealing membrane which excludes microbial contaminants but functionally permeable to oxygen and carbon dioxide gases.

U.S. Pat. No. 6,521,451 to Potter relates to a gas permeable cover for a cell culture. Further the permeable section is substantially impermeable to water and water vapor.

U.S. Pat. No. 8,053,230 to Whittlinger relates to a dish for culturing cells or tissue, wherein the dish comprises a channel formed on the bottom wall to minimize the effect of fluid movement on biologics in the dish. Further the dish comprises a barrier wall to provide a plurality of compartments to culture one or more biologics at a time. The barrier wall may also provide fluid communication between compartments.

It is apparent now that numerous innovations that are adapted to a variety of microbial culture devices like Petri dishes have been developed in the prior art that are adequate for various purposes. Furthermore, even though these innovations may be suitable for the specific purposes to which they address, accordingly, they would not be suitable for the purposes of the present invention as heretofore described. Thus, a resealable microbial culture and observation device that allows growth, transportation, and inspection of microbial growth characteristics is needed.

### SUMMARY OF THE INVENTION

The present invention relates to a resealable microbial culture and observation device that allows growth, transportation, and inspection of microbial growth characteristics. The device provides a tray for culturing a biologic, wherein the tray comprises at least one growth medium depression for containing a growth medium and a biologic, at least one anerobic member depression for containing an anerobic member that creates an anerobic environment for the biologic, and a vent to enable filtered gas exchange with the ambient environment, so as to supplement the microbial growth conditions in the growth medium depression; and further having a resealable label with a nontransparent portion for labeling, and a transparent portion for viewing the biologic.

According to an aspect of the present invention, a microbial culture and observation device comprises: a tray, wherein the tray includes a growth side, the growth side is defined by at least one growth medium depression, at least one anerobic member depression, multiple projections and at least an air vent; a filter mounted to the air vent, wherein the air vent forms a tunnel that is exposed to the ambient environment; and a sealing member, whereby the sealing member is configured to detachably attach to seal the growth side of the tray to create an anaerobic environment, further the projections are configured to create a passage to allow filtered fluid exchange between the anerobic environment and the ambient environment.

According to another aspect of the present invention, a sidewall and one or more supporting walls extends between the growth side and a mount side of the tray.

According to another aspect of the present invention, the growth medium depression is configured to contain a growth medium and a biologic.

According to another aspect of the present invention, the anerobic member depression is configured to contain a CO2 tablet that creates an anerobic environment for the biologic.

According to another aspect of the present invention, the sealing member is a label defined by an outer side and an inner side, the inner side of the label comprising an adhesive that facilitates to detachably attach to the growth side of the tray, the outer side is defined by a transparent portion and a nontransparent portion.

According to another aspect of the present invention, the label comprises a tab to enable gripping an edge of the label.

According to another aspect of the present invention, the filter that covers the vent at the growth side of the tray is a Tyvek® barrier.

According to another aspect of the present invention an objective of the present invention is to provide a tray that has standard (CLSI) dimensions for microbial testing.

Another objective is to create an anerobic environment between the growth surface of the tray and the sealing member.

Yet another objective is to ventilate the closed anerobic environment inside the tray, and the ambient environment outside of the tray.

Yet another objective is to provide notches that allow airflow among multiple stacked sealed tray in the incubator or during storage or transport, so as to save space.

Yet another objective is to allow medical related entities to grow, transport, and view a biologic, including clinical and veterinary diagnostic laboratories, clinical, industrial, and veterinary microbiology laboratories, biology research institutions, field epidemiology, environmental research laboratories, and educational teaching laboratories.

Yet another objective is to provide standard (CLSI) dimension for microbial testing including microbial growth inspection and antimicrobial resistance test without the need to open the device.

Yet another objective is to provide a resealable tray that allows for the transportation of inoculated clinical specimen in a sealed compartment and immediate incubation without further operations until microbial growth is observed thereby, saving operational time, cost and prevent exposure of personnel and environment to potential pathogens.

Yet another objective is to enable direct observation of microbial growth under the microscope without opening the seal, thereby minimizing cross contamination and exposure to microorganisms. For example, certain fungi species including dermatophytes can be viewed directly under the microscope for the hyphae characteristics.

Yet another objective is to provide a useful epidemiological tool for sample collection in the field.

Yet another objective is to provide a tray with ergonomic dimensions that enable an operator to manipulate the device with relative ease.

These and other objectives, advantages, and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The invention will now be described, by way of example, with reference to the accompanying drawings, in which:

FIG. 1 illustrates a perspective view of an exemplary resealable microbial culture and observation device, showing the growth side of the tray with the label removed, in accordance with an embodiment of the present invention;

FIG. 2 illustrates a perspective view of an exemplary resealable microbial culture and observation device shown in FIG. 1, showing the growth side of the tray with the label covering the medium depression and anerobic depression, in accordance with an embodiment of the present invention; and

FIG. 3 illustrates a perspective view of the resealable microbial culture and observation device shown in FIG. 1, showing the mount side of the tray, in accordance with an embodiment of the present invention.

Like reference numerals refer to like parts throughout the various views of the drawings.

### DETAILED DESCRIPTION OF THE INVENTION

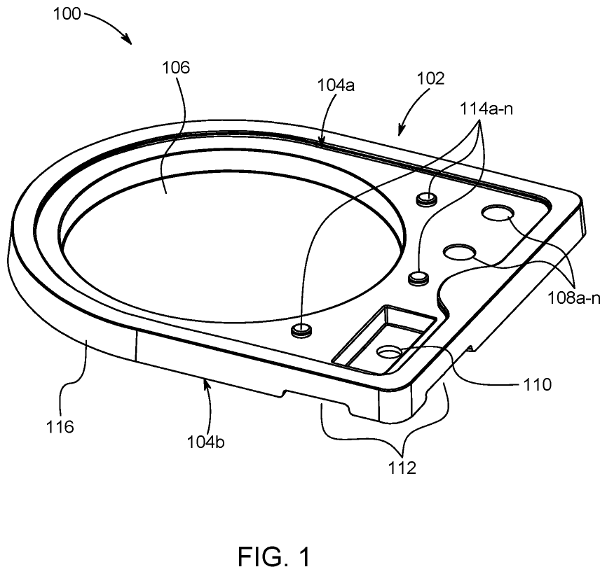

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIGS. 1-3. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting unless the claims expressly state otherwise.

A resealable microbial culture and observation device 100 is referenced in FIGS. 1-3. The resealable microbial culture and observation device 100, hereafter “device 100” provides a growth platform for the growth, transportation, and inspection of a biologic organism. FIG. 1 illustrates a perspective view of device 100, showing the components thereof that are sufficient just to describe the invention to a person skilled in the art.

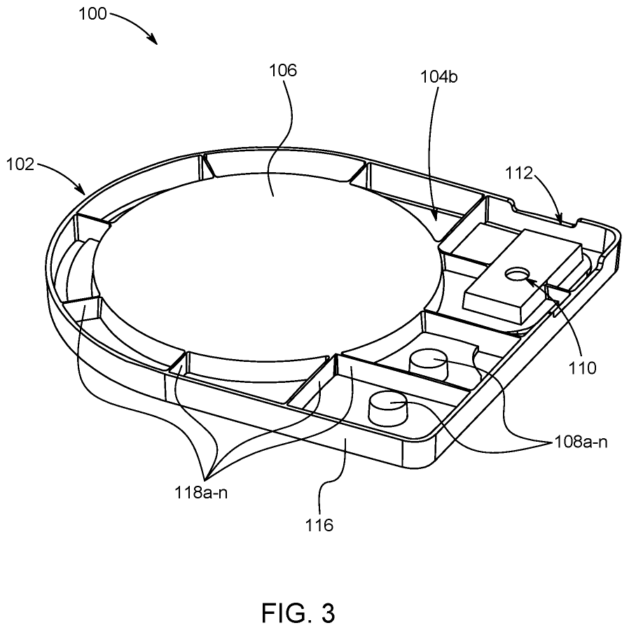

In some embodiments, the device 100 comprises a tray 102, upon which the growth, transport, and observation of the biologic occurs. The tray 102 is defined by at least one growth medium depression 106 that is sized and dimensioned to contain a growth medium. The growth medium is used to grow a biologic and do so in an anerobic environment. The biologic may include, without limitation, an organism that grows in an anerobic environment. The tray 102 is also defined by at least one anerobic member depression 108a-n that is sized and dimensioned to contain an anerobic member. The anerobic member creates an anerobic environment for the biologic inside the growth medium depression 106. Additionally, an air vent 110 forms in the tray 102. Air vent 110 is disposed through the tray and in fluid communication with a tunnel 112 beneath the tray as shown in FIG. 3. The tunnels 112 are configured to enable filtered gas exchange between the anerobic environment and an ambient environment. The ventilation through the air vent 110 serves to supplement the microbial growth conditions in the growth medium depression 106.

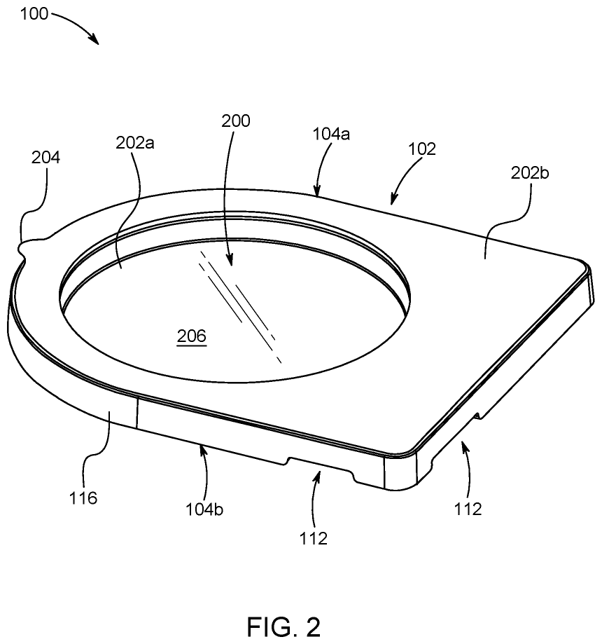

In other embodiments as shown in FIG. 2, a resealable sealing member 200 such as a label detachably covers the growth side 104a of the tray 102. The sealing member 200 is sized and dimensioned to selectively cover the growth medium depression 106 and the anerobic member depression 108a-n. This can be accomplished by overlaying the sealing member 200 over the growth side of the tray 102 and forming a bond between an adhesive seal in the label, and platforms and the periphery of the tray 102. The sealing member 200 has a nontransparent portion 202b for labeling, and a transparent portion 202a for viewing the biologic. As shown in FIG. 1, multiple platforms 114a-n extend from the growth side 104a of the tray 102 to elevate the sealing member 200 over the growth medium depression 106, and thereby increase airflow over the growth medium and the biologic.

According to an aspect of the present invention, a microbial culture and observation device 100, the device 100 comprises: a tray 102, wherein the tray 102 comprises a growth side 104a, the growth side 104a is defined by at least one growth medium depression 106, at least one anerobic member depression 108a-n, multiple projections 114a-n and at least an air vent 110; a filter (not shown) mounted to the air vent 110, wherein the air vent 110 forms a tunnel 112 that is exposed to the ambient environment; and a sealing member 200, whereby the sealing member 200 is configured to detachably attach to seal the growth side 104a of the tray 102 to create an anaerobic environment, further the projections 114a-n are configured to create at least one passage to allow filtered fluid exchange between the anerobic environment and the ambient environment.

In another aspect, a resealable microbial culture and observation device 100, comprises: a tray 102 comprising a growth side 104a and a mount side 104b with a sidewall 116 extending therebetween, the growth side 104a defined by at least one growth medium depression 106 for containing a growth medium and a biologic, the growth side 104a further being defined by at least one anerobic member depression 108a-n for containing an anerobic member that creates an anerobic environment for the biologic, the growth side 104a further forming an air vent 110 in fluid communication with an ambient environment, the air vent 110 forming a tunnel 112 along the mount side 104b of the tray 102, whereby filtered gas exchange exists between the anerobic environment and the ambient environment; a sealing member for example a label 200 defined by an outer side 206 (shown in FIG. 2) and an inner side, the inner side of the label 200 comprising an adhesive, whereby the label 200 detachably attaches to the growth side 104a of the tray 102, a portion of the label 200 being transparent to enable viewing the biologic, a portion of the label 200 being nontransparent to enable labeling the tray 102; and multiple platforms/projections 114a-n projecting from the growth side 104a of the tray 102, the platforms 114a-n operable to elevate the label 200 over the growth medium depression 106.

In another aspect, a resealable microbial culture and observation device, 100 the device 100 comprises: a tray 102, wherein the tray 102 comprises a growth side 104a, a mount side 104b, a sidewall 116 and one or more supporting walls 118a-n; the growth side 104a is defined by at least one growth medium depression 108 that is configured to contain a growth medium and a biologic; at least one anerobic member depression 108a-n on the growth side 104a of the tray 102, wherein the anerobic member depression 108a-n is configured to contain an anerobic member that creates an anerobic environment for the biologic; a filter (not shown) mounted to an air vent 110 at the growth side 104a of the tray 102, wherein the air vent 110 forms a tunnel 112 along the mount side 104b of the tray 102 to allow filtered fluid exchange between the anerobic environment and the ambient environment; a sealing member 200 defined by a transparent portion 202a and a nontransparent portion 202b, whereby the sealing member 200 detachably attaches to seal the growth side 104a of the tray 102; and multiple projections 114a-n from the growth side 104a of the tray 102, the projections 114a-n elevate the sealing member 200 over the growth medium depression 106 to create a passage for the fluid flow.

In another aspect, a portable and resealable microbial culture and observation device 100, the device 100 comprises: a U-shaped tray 102, wherein the tray 102 comprises a growth side 104a, a mount side 104b, a sidewall 116 and one or more supporting walls 118a-n; the growth side 104a defined by at least one growth medium depression 106 that is configured to contain a growth medium and a biologic; at least one anerobic member depression 108a-n on the growth side 104a of the tray 102, wherein the anerobic member depression 108a-n is configured to contain a CO2 generating member to creates an anerobic environment for the biologic; a filter (not shown) mounted to an air vent 110 at the growth side 104a of the tray 102, wherein the air vent 110 forms a tunnel 112 along the mount side 104b of the tray 102 to allow filtered fluid exchange between the anerobic environment and the ambient environment, further the tunnel 112 is configured facilitate stacking of the trays 102 and allows the filtered gas exchanges during the stacked condition; a sealing member 200, wherein the sealing member 200 is defined by an outer side 206, an inner side (not shown) and a tab 204, the inner side of the sealing member 200 comprises an adhesive, the outer side 206 is defined by a transparent portion 202a and a nontransparent portion 202b, the transparent portion 202a facilitates viewing the biologic whereas the nontransparent portion 202b facilitates to enable labeling the tray 102, further the tab 204 facilitates to grip the sealing member 200 to detachably attach it to the growth side 104a of the tray 102; and multiple projections 114a-n from the growth side 104a of the tray 102, the projections 114a-n elevate the sealing member 200 over the growth medium depression 106 and the anerobic member depression 108a-n to create a passage for the fluid flow.

In another aspect, the sidewall 116 and the one or more supporting walls 118a-n extending between the growth side and a mount side of the tray to provide strength and support to the tray 102.

In another aspect, the tray 102 is defined by a substantially U-shaped, however other shape and design of the tray 102 can be used without departing from the scope and spirit of the present invention.

In another aspect, multiple tray 102s are stackable, wherein the design of the sidewall 116, the supporting walls 118a-n, and the tunnel 112 facilitates easy and quick stacking of the trays for stowage and transport.

In another aspect, the tunnel 112 at the mount side of the tray 104b allows the filtered gas exchanges during the stacked condition.

In another aspect, the growth medium depression 106 is defined by a concave shape having diameter in the range 50-200 millimeter, however in a preferred embodiment the growth medium depression is 90-millimeter diameter. Though any other shape and dimension of the growth medium depression 106 can be used without departing from the scope and spirit of the present invention.

In another aspect, the sealing member 200 is a label defined by an outer side 206 and an inner side (not shown), the inner side of the label 200 comprising an adhesive along the periphery such that it facilitates to detachably attach to the growth side 104a of the tray 102 along the periphery of the sidewall 116.

In another aspect, the anerobic member comprises a CO₂ tablet.

In another aspect, the label 200 comprises at least one tab 204 to enable gripping edge of the sealing member 200.

In another aspect, the device 100 further comprises a Tyvek® barrier that covers the vent to function as a filter.

In another aspect, the tray 102 is fabricated from a medical grade polymer, glass, metal, metal alloy or a combination thereof.

As shown in FIG. 1, device 100 comprises a tray 102 that serves as a microbial culture device like a petri dish. The tray 102 is configured for growing a biologic, or performing bioassay, as known in the art. In some embodiments, the tray 102 has a growth side 104a on which the growth and required environment occur; and an opposing mount side 104b that rests flat on the surface, such as a laboratory bench. Both sides are parallel to each other with indentations, depressions, and cutouts on the growth side 104a being visible conversely on the mount side 104b. For example, the difference between the growth side 104a shown in FIG. 1, and the opposite mount side 104b of the tray 102 shown in FIG. 3 is clearly illustrated.

In some embodiments, a sidewall 116 extends between the growth side 104a and the mount side 104b. The sidewall 116 may be a few millimeters in height such that the tray 102 is small and compact, suitable for portability and stacking. The tray 102 may also have a generally U shape. In other embodiments, the tray 102 is generally flat and elongated, and could be rectangular in shape, however other shapes and designs may be used without departing from the scope and spirit of the present invention. The configuration of the device 100 is such that multiple trays 102 are stackable. This allows for easy transport and stowage, wherein the design of the sidewall 116, the supporting walls 118a-n (shown in FIG. 3), and the tunnel 112 on the mount side 104b of the tray 102 facilitates easy and quick stacking of the trays 102 for stowage and transport. Further as shown in FIG. 3, the sidewall 116 and the supporting walls 118a-n extending between the growth side and a mount side of the tray provides strength and support to the tray 102.

In this manner, the tray 102 enables transportation of inoculated clinical specimen in a sealed compartment and immediate incubation without further operations until microbial growth is observed thereby, saving operational time, cost and prevent exposure of personnel and environment to potential pathogens and contaminations. In one non-limiting embodiment, the tray 102 is fabricated from a medical grade polymer, glass, metal, metal alloy or a combination thereof. In other embodiments, different materials known in the art of bench chemistry, biological dishes, and Petri dishes may also be used. The tray 102 is configured to be reused and is also disposable.

In some embodiments, the growth side 104a of the tray 102 is defined by at least one growth medium depression 106 that is sized and dimensioned to receive and contain a growth medium used to grow the biologic. In some embodiments, the growth medium depression 106 is defined by a circular and/or concave shape having a 90-millimeter diameter. The shape of the growth medium depression 106 could be segmented to accommodate different formulations of biologically relevant media. Also, the diameter of the circular growth medium depression 106 receptacle may vary to accommodate differences in the overall size of the device 100. The growth medium depression 106 may, however, have other shapes, depths, or may be segregated into multiple separate depressions without departing from the scope and spirit of the present invention.

The growth side 104a further is defined by at least one anerobic member depression 108a-n for containing at least an anerobic member. The anerobic member is configured to create a CO₂ enriched atmosphere inside the tray 102, and specifically inside the growth medium depression 106. This produces an anerobic environment, which helps grow the biologic. Those skilled in the art will recognize that an anerobic organism is an organism that does not require oxygen for growth. Thus, in this case, the biologic is an anerobic organism. In one possible embodiment, the anerobic member comprises a CO₂ tablet. Other anaerobic means may also be used without departing from the scope and spirit of the present invention.

In yet another embodiment, the growth side 104a of the tray 102 forms an air vent 110 that is in fluid communication with an ambient environment, such as outside the tray 102. The air vent 110 serves as a tunnel 112; of sorts that allows air flow beneath the tray 102 to the interior space. The tunnel 112 forms at the mount side 104b of the tray 102. In this manner, filtered gas exchange exists between the anerobic environment and the ambient environment.

In one embodiment, the device 100 comprises a Tyvek® barrier that covers the vent 110 and helps in the filtration of the air/gas going into or leaving the tray 102. The Tyvek® barrier helps filter the airflow, however other such known filters may be used without departing from the scope and spirit of the present invention. In one exemplary use, multiple air vent 110s (notches) around the edges of the tray 102 enable optimal airflow into the Tyvek® barrier covered air vent 110, and other tray 102s stacked in an incubator. In any case, the air vent 110 allows for fresh air to enter the sealed anerobic environment of the tray 102, which stimulates the growth of the biologic in the growth medium.

Turning now to FIG. 2, the device 100 also provides a resilient, thin paper-like sealing member (also referred as a label) 200 that is configured to seal the anerobic environment, on the growth side 104a of the tray 102, and specifically in the growth medium depression 106 where the biologic rules. When the label 200 is sealed, the tray 102 helps prevent the spread of a pathogen or exposure of the specimen to the environment. The biologic specimen can also be transported more easily and safely when the growth side 104a of the tray 102 is sealed by the label 200. In one embodiment, the label 200 is fabricated from resilient materials that provide a bond with the platforms 114a-n extending from the growth side 104a of the tray 102. This bond forms a seal that covers the top of the platform. In one non-limiting embodiment, the label 200 is a resealable adhesive seal which doubles as a lid. Other known methods of sealing a member that acts as a label 200 over the growth side of the tray of the present invention may be used without departing from the scope and spirit of the present invention.

The label 200 is defined by an outer side that faces away from the growth side 104a of the tray 102, and an inner side that faces the growth side 104a of the tray 102. In some embodiments, the inner side of the label 200 has at the edges coated with an adhesive layer that enables the label 200 to temporarily stick perimeter of the tray 102. The adhesive enables the label 200 to detachably attach to the growth side 104a of the tray 102. In this manner, the label 200 can easily overlay growth side 104a of the tray 102, so as to substantially cover the growth medium depression 106.

The label 200 may also be easily removed from the tray 102, with this occurring multiple times, such that the tray 102 can be reused. In one possible embodiment, the label 200 comprises a tab 204 to enable gripping an edge of the label 200. The tab 204 can extend from one corner or an edge of the panel tray 102 for opening and closing access to the growth medium depression 106 in the tray 102.

A transparent portion 202a of the label 200 is transparent. The unique transparent configuration of the label 200 allows the biologic during growth, or while performing procedures thereon. In one embodiment, the portion of the label 200 that is transparent is the portion that leaves the medium depression. In this manner, the biologic can be viewed through a microscope or by visual inspection.

Additionally, the label 200 includes a non-transparent, or opaque portion 202b that is used for marking and labeling the device 100. This allows the outer side of the label 200 to serve as a surface for marking. Those skilled in the heart will recognize that proper labeling is important during biological procedures. In this transparent/nontransparent configuration, the label 200 is transparent over the growth medium depression 106 containing growth medium, and nontransparent over the other section of the tray 102 enabling labeling of the tray 102.

In an exemplary embodiment a transparent glass plate (not shown) may be slidably mounted in airtight manner to the growth side 104a of the tray 102 to removably cover and seal the growth side 104a of the tray 102 to facilitate anerobic environment specifically in the growth medium depression 106 where the biologic is cultured or evaluated and observed. Further the top glass plate acts as a protecting cover for the device 100 when stacked for storage or transportation.

Looking again at FIG. 1, the device 100 provides multiple platforms or projections 114a-n that project from the growth side 104a of the tray 102. The platforms 114a-n work to lift the label 200 so that there is headspace above the growth side 104a of the tray 102; thereby elevating the sealing member 200 such as a label (shown in FIG. 2) over the growth medium depression 106. The inner side of the label 200 rests on the top of the platforms 114a-n. In another embodiment, the platforms 114a-n about the adhesive seal to facilitate airflow/gas-flow into the growth medium. In one embodiment, the platforms 114a-n have a circular shape, and are spaced apart across the width of the tray 102, so as to create a uniform passage for airflow/gas-flow into the growth medium.

In conclusion, the device 100 provides a unique tray 102 defined by a growth medium depression 106. The growth medium depression 106 is configured to receive and contain a growth medium used to grow a biologic. The tray 102 also has an anerobic member depression 108a-n for containing an anerobic member that creates an anerobic environment therein. A vent forms in the tray 102 to enable filtered gas exchange between the anerobic environment and ambient environment. The ventilation supplements microbial growth conditions inside tray 102. A resealable label 200 detachably covers the growth side 104a of the tray 102, covering the growth medium depression 106 and anerobic member depression 108a-n. The label 200 has a nontransparent portion 202b for labeling, and a transparent portion 202a for viewing the biologic. Multiple platforms in the form of projections 114a-n extend from the growth side 104a of the tray 102 to elevate the label 200 over the growth medium depression 106, so as to increase airflow over the biologic.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A microbial culture and observation device, the device comprises:
a tray, wherein the tray comprises a growth side, the growth side is defined by at least one growth medium depression, at least one anerobic member depression, multiple projections and at least an air vent;
a filter mounted to the air vent, wherein the air vent forms a tunnel that is exposed to the ambient environment; and
a sealing member, whereby the sealing member is configured to detachably attach to seal the growth side of the tray to create an anaerobic environment, further the projections are configured to create a passage to allow filtered fluid exchange between the anerobic environment and the ambient environment.

2. The device of claim 1, wherein the tray is portable and is rectangular in shape.

3. The device of claim 1, wherein a sidewall and one or more supporting walls extending between the growth side and a mount side of the tray.

4. The device of claim 1, wherein the growth medium depression is configured to contain a growth medium and a biologic.

5. The device of claim 1, wherein the anerobic member depression is configured to contain a CO₂ tablet that creates an anerobic environment for the biologic.

6. The device of claim 1, wherein the sealing member is a label defined by an outer side and an inner side, the inner side of the label comprising an adhesive that facilitates to resealably attach to the growth side of the tray, the outer side is defined by a transparent portion and a nontransparent portion.

7. The device of claim 5, wherein the label comprises a tab to enable gripping an edge of the label.

8. The device of claim 1, wherein the filter that covers the vent at the growth side of the tray is a Tyvek® barrier.

9. A resealable microbial culture and observation device, the device comprises:
a tray, wherein the tray comprises a growth side, a mount side, a sidewall and one or more supporting walls;
the growth side defined by at least one growth medium depression that is configured to contain a growth medium and a biologic;
at least one anerobic member depression on the growth side of the tray, wherein the anerobic member depression is configured to contain an anerobic member that creates an anerobic environment for the biologic;
a filter mounted to an air vent at the growth side of the tray, wherein the air vent forms a tunnel along the mount side of the tray to allow filtered fluid exchange between the anerobic environment and the ambient environment;
a sealing member defined by a transparent portion and a nontransparent portion, whereby the sealing member detachably attaches to seal the growth side of the tray; and
multiple projections from the growth side of the tray, the projections elevate the sealing member over the growth medium depression to create a passage for the fluid flow.

10. The device of claim 9, wherein the tray is portable in size and the tray is defined by a U-shape.

11. The device of claim 9, wherein the tray is fabricated from a material selected from the group consisting of medical grade polymer, glass, metal, metal alloy or a combination thereof.

12. The device of claim 9, wherein multiple trays are stackable, wherein the tunnel at the mount side of the tray allows the filtered gas exchanges during the stacked condition.

13. The device of claim 9, wherein the growth medium depression is defined by a concave shape having a diameter 50-200 millimeter.

14. The device of claim 9, wherein the anerobic member comprises a CO₂ tablet.

15. The device of claim 9, wherein the sealing member is a label defined by an outer side and an inner side, the inner side of the label comprising an adhesive that facilitates to detachably attach to the growth side of the tray.

16. The device of claim 15, wherein the label comprises a tab to enable gripping an edge of the label.

17. The device of claim 9, wherein the filter that covers the vent at the growth side of the tray is a Tyvek® barrier.

18. A portable and resealable microbial culture and observation device, the device comprises:
a U-shaped tray, wherein the tray comprises a growth side, a mount side, a sidewall and one or more supporting walls;
the growth side defined by at least one growth medium depression that is configured to contain a growth medium and a biologic;
at least one anerobic member depression on the growth side of the tray, wherein the anerobic member depression is configured to contain a CO₂ generating member to creates an anerobic environment for the biologic;
a filter mounted to an air vent at the growth side of the tray, wherein the air vent forms a tunnel along the mount side of the tray to allow filtered fluid exchange between the anerobic environment and the ambient environment, further the tunnel is configured facilitate stacking of the trays and allows the filtered gas exchanges during the stacked condition;
a sealing member, wherein the sealing member is defined by an outer side, an inner side and a tab, the inner side of the sealing member comprises an adhesive, the outer side is defined by a transparent portion and a nontransparent portion, the transparent portion facilitates viewing the biologic whereas the nontransparent portion facilitates to enable labeling the tray, further the tab facilitates to grip the sealing member to detachably attach it to the growth side of the tray; and
multiple projections from the growth side of the tray, the projections elevate the sealing member over the growth medium depression and the anerobic member depression to create a passage for the fluid flow.

19. The device of claim 18, wherein the growth medium depression is defined by a concave shape having a 90-millimeter diameter.

20. The device of claim 18, wherein the tray is fabricated from a material selected from the group consisting of medical grade polymer, glass, metal, metal alloy or a combination thereof.
